(19) United States
(12) Patent Application Publication
(10) Pub. No.: US 2024/0157099 A1
(43) Pub. Date: May 16, 2024

(54) MICRO BALLOON CATHETER WITH DISTAL VENT

(71) Applicant: Merit Medical Systems, Inc., South Jordan, UT (US)

(72) Inventors: Jason Wiersdorf, West Jordan, UT (US); Dillon Despain, Bluffdale, UT (US)

(21) Appl. No.: 18/506,802

(22) Filed: Nov. 10, 2023

Related U.S. Application Data
(60) Provisional application No. 63/383,331, filed on Nov. 11, 2022.

(51) Int. Cl. A61M 25/10; A61M 25/00; A61M 39/10

(52) U.S. Cl. CPC A61M 25/1018; A61M 25/005; A61M 39/10

(57) ABSTRACT

Devices and methods used to selectively occlude a blood vessel are disclosed. The devices include a catheter having a balloon disposed at a distal end and a connector coupled to a proximal end of the catheter. A handle is coupled to the connector. A slide actuator is disposed within the handle. A seal wire is coupled to the slide actuator. When the slide actuator is in a distal position, the seal wire seals a vent port distal of the balloon to prevent inflation fluid from flowing from the balloon. When the slide actuator is in a proximal position, the seal wire is displaced proximally to unseal the vent port and allow fluid to flow from the balloon and distally out of the vent port resulting in self-deflation of the balloon.

### RELATED APPLICATIONS

The present application claims priority to U.S. Provisional Application 63/383,331, filed on Nov. 11, 2022 and titled, “Micro Balloon Catheter with Distal Vent,” which is hereby incorporated by reference in its entirety.

### TECHNICAL FIELD

The present disclosure relates generally to devices to treat organs intravascularly. More specifically, the present disclosure relates to a micro balloon catheter device used to occlude a patient's blood vessel.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an embodiment of a micro balloon catheter device in an inflated state.

FIG. 2 is a cross-sectional side view of a proximal portion of the micro balloon catheter device of FIG. 1.

FIG. 3 is cross-sectional side view of a distal portion of the micro balloon catheter device of FIG. 1.

FIG. 3A is a cross-sectional view of the distal portion of FIG. 3 through section line 3A-3A.

FIG. 4A is a side view of the micro balloon catheter device of FIG. 1 inserted into a patient's blood vessel in a ready state.

FIG. 4B is a side view of a distal portion of the micro balloon catheter device of FIG. 4A.

FIG. 5A is a side view of the micro balloon catheter device of FIG. 1 inserted into a patient's blood vessel in an inflated state.

FIG. 5B is a side view of a distal portion of the micro balloon catheter device of FIG. 5A.

FIG. 6A is a side view of the micro balloon catheter device of FIG. 1 inserted into a patient's blood vessel in a deflated state.

FIG. 6B is a side view of a distal portion of the micro balloon catheter device of FIG. 6A.

### DETAILED DESCRIPTION

In certain instances, a diseased patient organ can be treated intravascularly using targeted delivery of a treatment substance. For example, an organ having a tumor or cancerous growth may be treated by intravascular targeted delivery of a chemotherapeutic drug. In another example, a blood vessel within an organ can be permanently occluded to prevent blood flow into a tumor by targeted delivery of a thrombogenic agent. The intravascular treatment can utilize a micro balloon catheter to occlude the blood vessel distal of the treatment site and prevent flow of the treatment substance from the treatment site.

Embodiments herein describe micro balloon catheter devices and methods to assist in targeted intravascular delivery of a treatment substance. The device can be percutaneously inserted into a blood vessel of the organ or body area to be treated. In some embodiments within the scope of this disclosure, the devices include an elongate catheter or tube coupled to a connector having an inflation port. An inflation or expandable member (e.g., balloon) is disposed adjacent a distal end of the catheter. The balloon is in fluid communication with the inflation port through the catheter wherein the balloon can be inflated or expanded with a fluid. A vent port is coupled to the distal end of the catheter. A bore extends through the vent port and is in fluid communication with the balloon. The bore has a proximal portion and a distal portion. A diameter of the proximal portion is larger than a diameter of the distal portion. The bore has a distal opening.

A handle or actuator is couplable to the connector. The handle includes a slide actuator disposed within a housing. The slide actuator is displaceable between a distal position and a proximal position. A seal wire is coupled to the slide actuator and is axially displaceable by the slide actuator. When the slide actuator is in the distal position, a distal end of the seal wire is sealingly disposed within the distal portion of the bore of the vent port to prevent fluid from flowing from the balloon and out the opening. This configuration allows the balloon to be inflated or expanded by the fluid delivered from the inflation port. When the slide actuator is in the proximal position, the distal end of the seal wire is disposed within the proximal portion of the bore of the vent port. This configuration allows fluid from the balloon to flow through the bore and out the opening resulting in self-deflation of the balloon within a short period of time.

In use, in embodiments within the scope of this disclosure, the micro balloon catheter device is percutaneously inserted into the blood vessel of the organ to be treated such that the distal end of the micro balloon catheter device is positioned adjacent a desired treatment site. When inserted, the micro balloon catheter device is in a ready state where the balloon is deflated, the slide actuator is in the distal position, and the distal end of the seal wire is sealingly disposed within the distal portion of the bore of the vent port. The balloon may be inflated or expanded by injection of a fluid from a fluid delivery device coupled to the inflation port. The fluid can flow through the inflation port, through an annular space within the catheter defined by the seal wire and the catheter wall, through a side port of the catheter, and into the balloon. The seal of the seal wire within the distal portion of the bore prevents fluid from flowing out the distal opening of the vent port. The balloon may be deflated by displacement of the slide actuator to the proximal position resulting in proximal displacement of the seal wire from within the distal portion to the proximal portion of the bore of the vent port. This configuration allows fluid to flow from the balloon, through the side port, distally through the annular space, through the proximal portion around the seal wire, through the distal portion, and out the distal opening resulting in self-deflation of the balloon within a short time period of one to five seconds.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

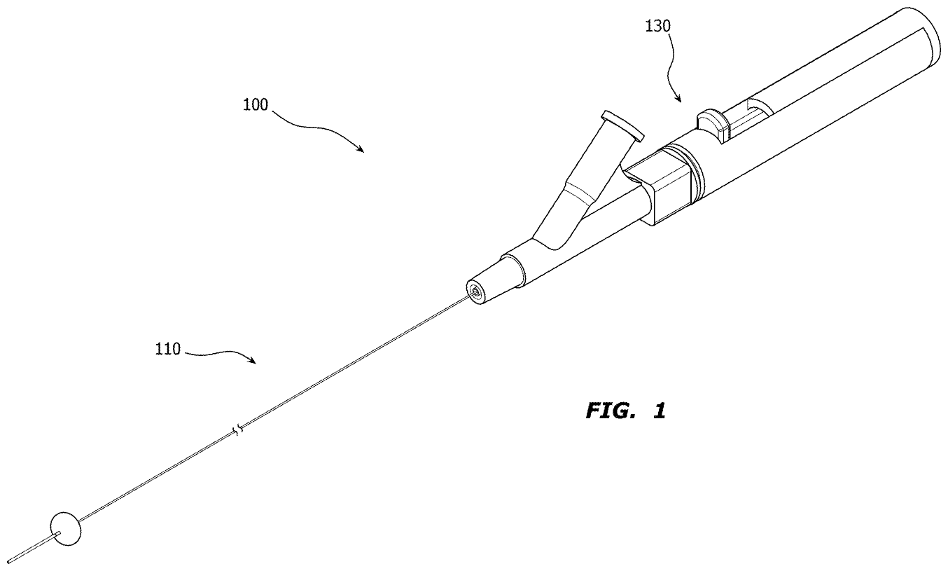

FIGS. 1-6B illustrate an embodiment of a micro balloon catheter device and embodiments of its various components. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

As illustrated in FIG. 1, a micro balloon catheter device 100 includes two broad groups of components; each group may have numerous subcomponents and parts. The two broad component groups are: a micro catheter assembly 110 and a handle or actuator assembly 130.

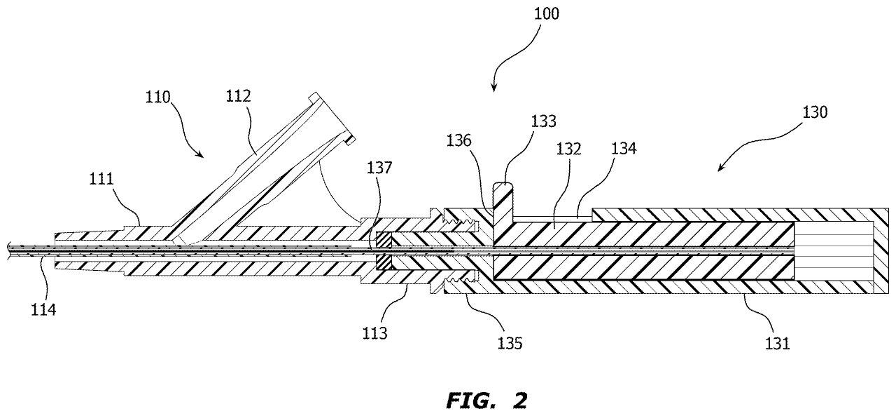

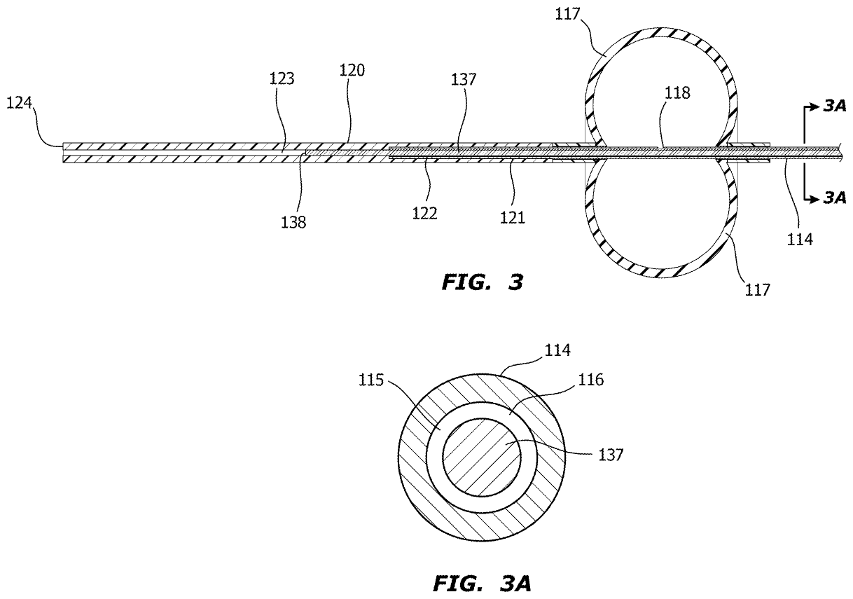

As depicted in FIGS. 2, 3, and 3A, the micro catheter assembly 110 includes a connector 111, an elongate tube or catheter 114, an expandable or inflatable vessel occlusion member 117, and a vent port or fluid evacuation port 120. The connector 111 is coupled to a proximal portion of the catheter 114 and may have a “Y” shape. An inflation port 112 is in fluid communication with a lumen 115 of the catheter 114 wherein a fluid can be injected from a fluid delivery device (e.g., syringe) coupled to the inflation port 112 to inflate the inflatable or expandable vessel occlusion member 117. In certain embodiments, the connector 111 is fixedly coupled to the catheter 114. In other embodiments, the connector 111 may be selectively coupled to the catheter 114, wherein the connector 111 may be decoupled from the catheter 114 to allow placement of a catheter, such as an infusion catheter, over the catheter 114 to accomplish a desired medical procedure.

The catheter 114 extends distally from the connector 111. The catheter 114 may formed of a shape memory metal alloy, such as nitinol, or any other suitable material that allows the catheter 114 to be maneuvered through a tortuous vessel path. An outer diameter of the catheter 114 may range from about 0.25 millimeters to about 0.5 millimeters, and may be about 0.27 millimeters. The lumen 115 extends through the catheter 114 in alignment with a longitudinal axis of the catheter 114. A diameter of the lumen 115 may range from about 0.18 millimeters to about 0.35 millimeters, and may be about 0.18 millimeters.

As shown in FIG. 3, the expandable or inflatable vessel occlusion member 117 is coupled to the catheter 114 adjacent a distal end using any suitable technique, such as welding, bonding, gluing, etc. The occlusion member 117 may be a balloon formed of any suitable elastomeric material configured to elongate and withstand rupturing at a fluid pressure ranging from about 5 pounds per square inch to about 24 pounds per square inch. For example, the occlusion member 117 may be formed of silicone, polyisoprene or an aromatic polyether based urethane and a styrene blocked copolymer. The occlusion member 117 is configured to have a deflated or non-expanded state wherein a diameter of the occlusion member 117 is substantially equivalent to the outer diameter of the catheter 114 in an inflated or expanded state, as shown in FIG. 3, wherein the diameter of the occlusion member 117 may range from about 0.5 millimeters to about 5 millimeters dependent upon the material, wall thickness, and fluid pressure applied to the occlusion member 117. When in the inflated state, the occlusion member 117 can occlude or block blood flow within a blood vessel as the occlusion member 117 engages with the wall of the blood vessel. In another embodiment, when in the inflated state, the occlusion member 117 may anchor the catheter 114 in place relative to the blood vessel as an infusion catheter is placed over the catheter 114. One or more additional catheters or devices may be advance along the catheter 114 or other portions of the assembly when the catheter 114 is so anchored.

The catheter 114 includes a side port 118 disposed through the wall of the catheter 114 and positioned within the occlusion member 117 to provide fluid communication between the lumen 115 and the occlusion member 117. When the occlusion member 117 is inflated or expanded, fluid flows through the inflation port 112, through the lumen 115, through the side port 118, and into the occlusion member 117.

The vent port 120 is coupled to a distal end of the catheter 114 and disposed distally of the occlusion member 117. The vent port 120 may be formed of any suitable polymeric material, such as pebax or nylon. In some embodiments, the vent port 120 may be flexible to permit passage of the catheter 114 through a tortuous vessel. In another embodiment, the vent port 120 and the catheter 114 may include a steering wire, such as a flat wire, extending from the vent port 120, along the catheter 114, and to a steering mechanism (e.g., wire tensioning mechanism) disposed at a proximal end of the catheter 114. The steering wire and the steering mechanism may be utilized to deflect the vent port 120 into an arcuate shape to allow steering of the catheter 114 through the tortuous vessel. Coupling of the vent port 120 to the catheter 114 can be accomplished using any suitable manufacturing technique, such as welding, bonding, gluing, overmolding, etc. An outer diameter of the vent port 120 may be substantially equivalent to the outer diameter of the catheter 114.

A bore 121 extends longitudinally through the vent port 120. The bore 121 is in axial alignment and fluid communication with the lumen 115. The bore 121 includes a first or proximal portion 122 having a diameter ranging from about 0.18 millimeters to about 0.35 millimeters and a second or distal portion 123 which may have a smaller diameter. The bore 121 includes an opening 124 disposed at a distal end of the vent port 120. When the occlusion member 117 is deflated, the fluid within the occlusion member 117 can flow through the side port 118, through the lumen 115, through the proximal portion 122, through the distal portion 123, and through the opening 124 into the environment surrounding the vent port 120, such as the blood within the blood vessel.

As illustrated in FIGS. 2, 3, and 3A, the handle 130 includes a housing 131, a slider or slide member or actuator 132 slidingly disposed within the housing 131, and a seal wire 137. The slider 132 includes a tab 133 extending radially outward from a longitudinal axis of the slider 132. The tab 133 is disposed within a slot 134 of the housing 131. The slot 134 allows the slider 132 to be displaced between a distal position, as shown in FIG. 2, and a proximal position when a user engages the tab 133 with one or more fingers. The slot 134 includes a positive distal stop 136 to stop distal movement of the slider 132 relative to the housing 131. A distal end 135 of the housing 131 is couplable to a proximal end 113 of the connector 111. The distal end 135 may include internal threads to engage with external threads of the proximal end 113. In other embodiments, the housing 131 may be coupled to the connector 111 using any suitable technique, such as a snap fit, a friction fit, etc.

The seal wire 137 is operably coupled to the slider 132 and extends distally from the slider 132 through the connector 111. The seal wire 137 may be formed of any suitable material, such as stainless steel, shape memory metal alloy (e.g., nitinol), etc. A diameter of the seal wire 137 may range between about 0.13 millimeters and about 0.3 millimeters. When the slider 132 is displaced a distance proximally from the distal position, the seal wire 137 is displaced proximally an equal distance, and when the slider 132 is displaced a distance distally from the proximal position, the seal wire 137 is displaced distally an equal distance. The seal wire 137 is coaxially disposed within the lumen 115 of the catheter 114 to define an annular space 116 between the seal wire 137 and the wall of the catheter 114. A cross-sectional area of the annular space 116, may range from about 0.004 millimeters 2 to about 0.008 millimeters 2. The inflation port 112 and the occlusion member 117 are in fluid communication with the annular space 116 such that when the occlusion member 117 is inflated or expanded, fluid can flow through the inflation port 112, through the annular space 116, through the side port 118, and into the occlusion member 117.

As depicted in FIG. 3, the seal wire 137 extends into the bore 121 of the vent port 120. When the slider 132 is in the distal position, a distal portion 138 of the seal wire 137 is disposed within the second portion 123 and seals against an inner surface of the second portion 123 to prevent fluid from flowing through the bore 121 allowing the occlusion member 117 to be inflated and to maintain the inflated state. The positive distal stop 136 can prevent distal displacement of the seal wire 137 that results in the distal portion 138 extending distally from the vent port 120. When the slider 132 is in the proximal position, the distal portion 138 is disposed within the first portion 122 allowing fluid to flow from the occlusion member 117 as it deflates, through the side port 118, through the annular space 116, through the first portion 122 around the seal wire 137, through the second portion 123, and out the opening 124 into the environment surrounding the vent port 120.

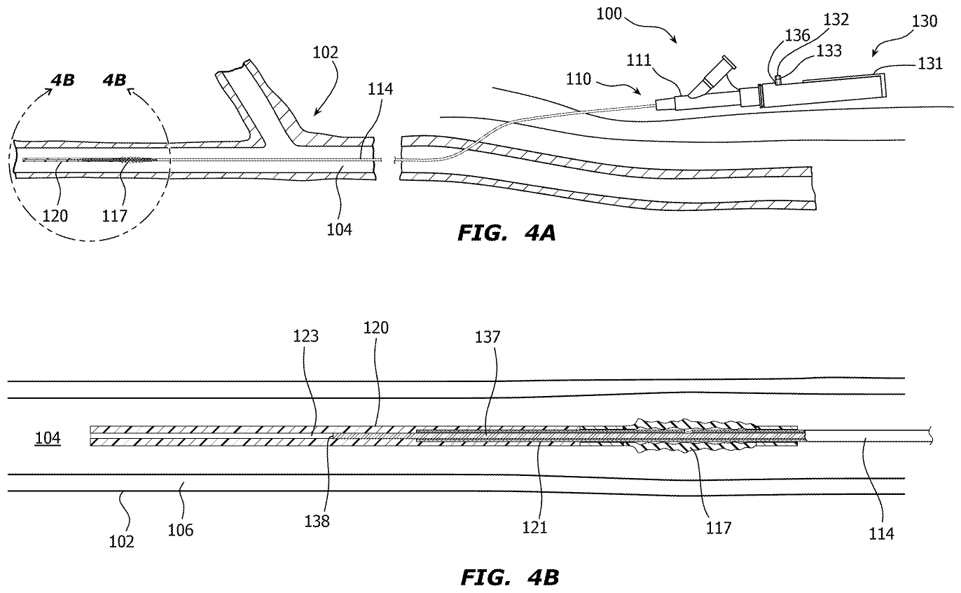

FIGS. 4A-6B illustrate the micro balloon catheter device 100 in use. As illustrated in FIGS. 4A and 4B, the micro balloon catheter device 100 is in a ready state and percutaneously inserted through a patient's skin into a lumen 104 of a patient's blood vessel 102. For example, the micro balloon catheter device 100 may be inserted into a blood vessel of a patient's liver, pancreas, neck, and leg. Other blood vessels are contemplated within the scope of this disclosure. The flexibility of the vent port 120 may allow the catheter 114 to be guided through tortuous vessels to a desired treatment location. In another embodiment, the micro balloon catheter device 100 may include a steering wire and a steering mechanism, as previously described, to help guide or steer the catheter 114 through the tortuous vessels.

The handle 130 is coupled to the connector 111 of the micro catheter assembly 110 with the slider 132 in the distal position wherein the tab 133 is disposed against the positive distal stop 136 of the housing 131 to position the distal portion 138 of the seal wire 137 within the distal portion 123 of the bore 121 of vent port 120. The vessel occlusion member 117 is in a deflated or non-expanded state wherein the outer diameter is substantially equivalent to the outer diameter of the catheter 114 and the vent port 120.

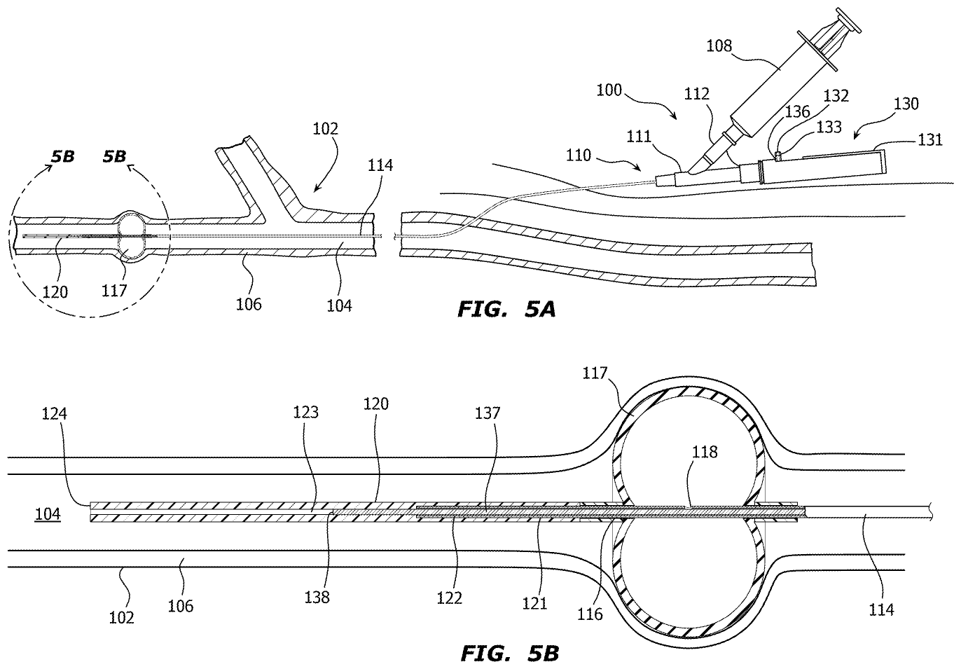

FIGS. 5A and 5B illustrate the micro balloon catheter device 100 in a vessel occluding state wherein the occlusion member 117 is inflated against a wall 106 of the vessel 102 to occlude or block blood flow through the lumen 104 of the vessel 102. The handle 130 is coupled to the connector 111 of the micro catheter assembly 110 with the slider 132 in the distal position wherein the tab 133 is disposed against the positive distal stop 136 of the housing 131 to position the distal portion 138 of the seal wire 137 within the distal portion 123 of the bore 121 of vent port 120. When disposed within the distal portion 123, the seal wire 137 seals the distal portion 123 to prevent or restrict fluid from flowing through the distal portion 123 when the occlusion member 117 is inflated or expanded with a fluid. The positive distal stop 136 may also prevent the seal wire 137 from extending distally from the opening 124 of the vent port 120.

A fluid delivery device 108 (e.g., syringe) is coupled to the inflation port 112 of the connector 111. The fluid delivery device 108 may be at least partially filled with any suitable fluid configured to inflate or expand the occlusion member 117. For example, the fluid may be saline, contrast media, a saline and contrast media mixture, etc. The fluid is injected into the micro balloon catheter device 100 by the fluid delivery device 108 wherein the fluid flows through the inflation port 112, the annular space 116, the side port 118, and into the occlusion member 117 resulting in inflation or expansion of the occlusion member 117. The occlusion member 117 may be inflated or expanded at a fluid pressure ranging from about 5 pounds per square inch to about 19 pounds per square inch.

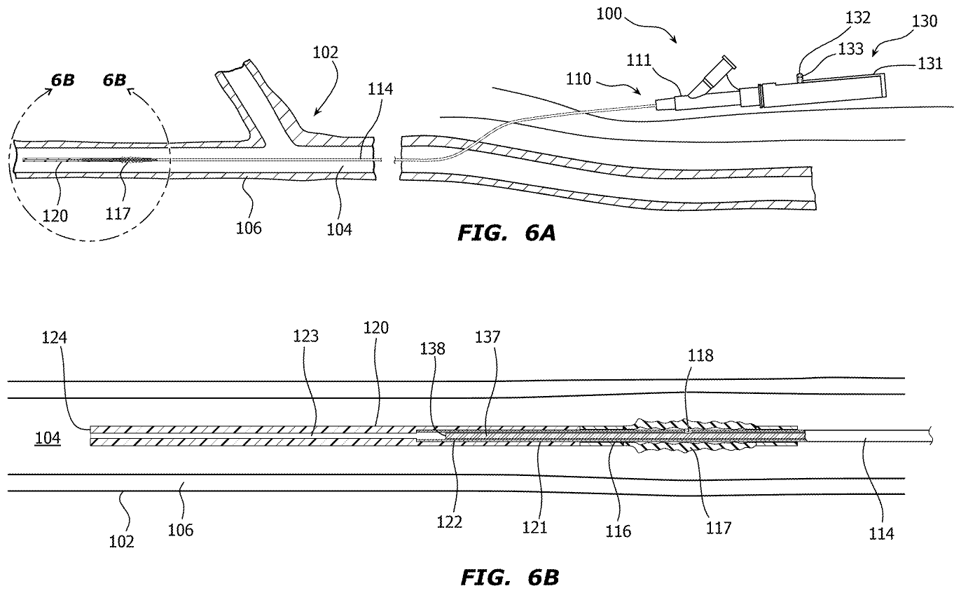

FIGS. 6A and 6B illustrate the micro balloon catheter device 100 in a deflated state where the occlusion member 117 is deflated to allow blood flow through the lumen 104 of the vessel 102. As illustrated, the handle 130 is coupled to the connector 111 of the micro catheter assembly 110. The slider 132 is displaced to the proximal position within the housing 131. The distal portion 138 of the seal wire 137 is displaced into the proximal portion 122 of the bore 121 of the vent port 120. When the distal portion 138 is disposed within the proximal portion 122, the distal portion 123 is open to allow fluid to flow from the occlusion member 117, through the side port 118, through the annular space 116, through the proximal portion 122 around the seal wire 137, through the distal portion 123, through the opening 124, and into the lumen 104 of the blood vessel 102. The fluid flow can be pressurized by the elastomeric contraction of the occlusion member 117 and may flow out the opening 124 due to less distal flow resistance than proximal flow resistance. The occlusion member 117 may self-deflate over a time ranging from about one second to about five seconds.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of occluding a vessel may include one or more of the following steps: disposing an actuator in a first position, wherein a seal member seals a bore of an occlusion member evacuation port of a vessel occlusion device; inflating an expandable occlusion member with a fluid to occlude a vessel; moving the actuator from the first position to a second position, wherein the occlusion member is displaced to open the bore; and deflating the expandable occlusion member, wherein the fluid flows from the expandable occlusion member into the bore and out a distal end of the occlusion member evacuation port. Other steps are also contemplated.

In the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest to the practitioner during use. As specifically applied to a micro balloon catheter device of this disclosure, the proximal end of the device refers to the end nearest to the handle and the distal end refers to the opposite end, the end nearest to the vent port.

“Fluid” is used in its broadest sense, to refer to any fluid, including both liquids and gases as well as solutions, compounds, suspensions, etc., which generally behave as fluids.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially equivalent” is recited with respect to a feature, it is understood that in further embodiments, the feature can have a precisely equivalent configuration.

The terms “a” and “an” can be described as one, but not limited to one. For example, although the disclosure may recite a housing having “a connector,” the disclosure also contemplates that the housing can have two or more connectors.

Unless otherwise stated, all ranges include both endpoints and all numbers between the endpoints.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A micro balloon catheter, comprising:
a catheter comprising:
a distal end portion comprising a vent port;
an expandable member disposed proximally to the vent port and in fluid communication with the vent port;
a connector coupled to the catheter;
a handle coupled to the connector, wherein the handle comprises a slide member moveable between a distal position and a proximal position; and
a wire coupled to the slide member and slidingly disposed within the catheter, wherein a distal portion of the wire is configured to seal the vent port when the slide member is in the distal position and to open the vent port when the slide member is in the proximal position.

2. The micro balloon catheter of claim 1, wherein the expandable member is inflatable with a fluid when the slide member is in the distal position.

3. The micro balloon catheter of claim 1, wherein the expandable member is deflatable when the slide member is in the proximal position.

4. The micro balloon catheter of claim 1,
wherein the vent port comprises a lumen comprising a first portion and a second diameter portion, wherein a diameter of the second portion is smaller than a diameter of the second portion,
wherein the distal portion of the wire is configured to seal the vent port when disposed within the second portion, and
wherein the distal portion of the wire is configured to open the vent port when disposed within the first portion.

5. The micro balloon catheter of claim 4, wherein the lumen comprises an open distal end configured to vent fluid from the inflatable member into an exterior environment when the distal portion of the wire is disposed within the first portion.

6. The micro balloon catheter of claim 1,
wherein the connector comprises an inflation port;
wherein the catheter comprises a side port disposed within the inflatable member;
wherein the catheter comprises an annular space disposed between an inner wall of the catheter and the wire and extending between the inflation port and the side port; and
wherein the inflation port is in fluid communication with the annular space and the annular space is in fluid communication with the inflatable member through the side port.

7. The micro balloon catheter of claim 6, wherein the vent port is in fluid communication with the inflatable member through the side port.

8. The micro balloon catheter of claim 6, wherein a cross-sectional area of the second portion is larger than the cross-sectional area of the annular space.

9. The micro balloon catheter of claim 1,
wherein the catheter comprises a shape memory metal alloy material, and
wherein the vent port comprises a polymeric material.

10. The micro balloon catheter of claim 1, wherein the expandable member is a balloon.

11. A vessel occlusion device, comprising:
an elongate tube comprising:
a fluid evacuation port;
a vessel occlusion member disposed proximally to the fluid evacuation port and in fluid communication with the fluid evacuation port;
a connector coupled to the elongate tube;
an actuator coupleable to the connector, wherein the actuator comprises a slider moveable between a first position and a second position; and
a sealing member coupled to the slider and slidingly disposed within the elongate tube, wherein a portion of the sealing member prevents fluid from flowing through the fluid evacuation port when the slider is in the first position and allows fluid to flow through the fluid evacuation port when the slider is in the second position.

12. The vessel occlusion device of claim 11,
wherein the vessel occlusion member is inflatable when the slider is in the first position, and
wherein the vessel occlusion member is deflatable when the slider is in the second position.

13. The vessel occlusion device of claim 11, wherein the fluid evacuation port comprises a bore comprising:
a first portion having a first diameter;
a second portion having a second diameter smaller than the first diameter; and
an open distal end.

14. The vessel occlusion device of claim 13,
wherein a distal end of the sealing member is disposed within the second portion when the slider is in the first position to seal the fluid evacuation port, and
wherein the distal end of the sealing member is disposed within the first portion when the slider is in the second position to open the evacuation port.

15. The vessel occlusion device of claim 14, wherein fluid is configured to flow from the vessel occlusion member, through the bore of the fluid evacuation port, and out the open distal end of the bore when the slider is in the second position.

16. A method of occluding a vessel, comprising:
disposing an actuator in a first position, wherein a seal member seals a bore of an occlusion member evacuation port of a vessel occlusion device;
inflating an expandable occlusion member with a fluid to occlude a vessel;
moving the actuator from the first position to a second position, wherein the seal member is displaced to open the bore; and
deflating the expandable occlusion member, wherein the fluid flows from the expandable occlusion member into the bore and out a distal end of the occlusion member evacuation port.

17. The method of claim 16, further comprising anchoring the vessel occlusion device relative to the vessel when the expandable occlusion member is inflated.

18. The method of claim 16, further comprising disposing an infusion device over the vessel occlusion device.

19. The method of claim 16, further comprising preventing extending the seal member from the occlusion member evacuation port when the actuator is in the first position.

20. The method of claim 16, wherein deflating the expandable occlusion member comprises a deflation time of between one second and five seconds.
